(19) United States
(12) Patent Application Publication
(10) Pub. No.: US 2026/0345845 A1
(43) Pub. Date: Nov. 5, 2026

(54) LARGE ATMOSPHERIC PRESSURE PLASMA ARRAY

(71) Applicant: Boise State University, Boise, ID (US)

(72) Inventors: Jim Browning, Boise, ID (US); Ranajoy Bhattacharya, Boise, ID (US); Robert Hay, Boise, ID (US); Ken Cornell, Boise, ID (US)

(21) Appl. No.: 19/664,562

(22) Filed: Apr. 30, 2026

Related U.S. Application Data
(60) Provisional application No. 63/797,596, filed on Apr. 30, 2025.

(51) Int. Cl. A61L 2/14; A61L 2/26; B01J 19/08

(52) U.S. Cl. CPC A61L 2/14; A61L 2/26; B01J 19/088; A61L 2202/11; B01J 2219/0809; B01J 2219/0835; B01J 2219/0841; B01J 2219/0869; B01J 2219/0896

(57) ABSTRACT

This disclosure relates to Cold Atmospheric Pressure Plasma (CAP) array systems for plasma treatment over large surface areas (>100 cm2). Specifically, the array comprises stacked discharge plasma elements (plates) configured for a working gas to flow through the gaps created in the array of discharge plates. In some embodiments, the system further comprises a gas capture system to collect plasma gases, a recirculating system to redirect the plasma species back into the array, a showerhead for gas uniformity, a filter system, and/or a cooling system.

### CROSS-REFERENCE TO RELATED APPLICATIONS

This application claims priority under 35 U.S.C. § 119 to provisional application Ser. No. 63/797,596, filed Apr. 30, 2025, herein incorporated by reference in its entirety.

### STATEMENT REGARDING FEDERALLY SPONSORED RESEARCH OR DEVELOPMENT

This invention was made with government support under #1R21EB031257-01 awarded by the National Institute of Health (NIH NIBIB) and #2020-67018-30789 awarded by the US Department of Agriculture NIFA. The government has certain rights in the invention.

### TECHNICAL FIELD

The present disclosure relates generally to a Cold Atmospheric Pressure Plasma (CAP) array system for plasma treatment over large surface areas (>100 cm2). Specifically, the array is created by stacking linear discharge plasma elements together in an enclosure so that air or other gas will flow through the gaps created in the array of discharge plates. More particularly, but not exclusively, the present disclosure describes the array, the enclosure, an electrical connection structure, a gas driver, a gas capture system to collect plasma gases, a recirculating system to redirect the plasma species back into the array, a showerhead for gas uniformity, a filter system, and a cooling system.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Poor sanitization and control of microbial pathogens in food processing contribute to the ~48 million cases of foodborne illness that lead to about 128,000 hospitalizations in the U.S. each year. Losses in crop production due to plant diseases and pests cost the global economy an estimated $220 billion annually. Thus, the safety of our national food supply and the sustainability of our crop production is under constant threat by the ubiquitous presence of viral, bacterial, and fungal pathogens that cause human foodborne illness and plant diseases that decrease crop yields. These pathogens may be present in or on the raw foodstuffs or crop seed, or be introduced during processing. In addition, in food processing plants, microbes can contaminate equipment surfaces and form biofilms that are difficult to eradicate, in turn serving as a reservoir for organisms that lead to food spoilage and foodborne illness in consumers.

Biofilms are a major source of spoilage and threaten the quality and safety of food products that come in contact with these surfaces. Biofilms also develop rapidly and their growth on conveyors and other food-contact surfaces can contaminate food products even to the level of >10 million bacteria per ml in some contexts. These biofilms also impact food processing operations, impairing proper equipment performance and requiring food-contact surfaces to be routinely cleaned using harsh chemicals and large volumes of water to remove chemical residues prior to re-contact with food products. Frequent sampling is often required to determine contamination levels that govern cleaning rates and remediation strategies. Food products and crop seed must also be treated to inactivate or remove pathogens.

The food processing industry spends significant time and expense to maintain the safety of food products. To maintain the quality and safety of these products, microbiological guidelines are an essential requirement. Biofilms can also be very difficult to detect. As part of a sanitation verification activities, facilities use swabs to test the surface of transport systems and perform agar plating to enumerate the microbial load. However, some of the biofilms formed by foodborne pathogens and other microbes can enter a “viable but non-culturable” status that complicates the evaluation of sanitization. Food processing factories routinely shut down for cleaning processes that requires flushing of surfaces with harsh chemicals and vast amounts of water. Biofilms in food processing continue to be costly, requiring additional sanitation that involves increased chemical and water usage and prolonged equipment downtime, as well as added product waste because of food safety or quality concerns. Biofilms have been found to be 10 to 1000 times more resistant to the sanitizers routinely used in a sanitation system (sodium hypochlorite, peroxides, quaternary ammonium compounds).

Food products, including all types of fresh produce, may also carry pathogens introduced in the fields during growth or harvest, that later contaminate processing facilities and/or transmit foodborne illness to consumers. In addition, crops raised as seeds for subsequent planting cycles may carry plant viral, bacterial, and fungal pathogens that decrease their storage lifespan, reduce germination efficiency, decrease crop yields, and serve as a source of transmission of disease to new fields. Ultimately, the carriage of these pathogens on seeds reduces their economic value as a crop and prevents their sale in certain markets.

Cold atmospheric-pressure plasma (CAP) has been studied as a sanitation method in medicine and the food processing industries. Many research efforts have proven the effectiveness of small CAP devices in inactivating and removing pathogens from surfaces and wounds. CAP devices do not rely on high temperatures but rather on reactive oxygen and nitrogen gas species to sanitize surfaces. This feature makes them safer for various applications, including sterilizing medical equipment, treating wounds, decontaminating food, and removing pathogens from surfaces. One of the most advantageous aspects of CAP devices is their ability to sanitize equipment and surfaces without resorting to harsh chemicals, which might leave harmful chemical residues on equipment and surfaces. However, these small CAP devices (i.e. 2.45 cm×2 cm active plasma region or less) limits their practicality for sanitizing large surface areas.

Therefore, there is a need for larger scale CAP devices for sanitizing surfaces and treating crop seeds and other produce to reduce cost, chemical, and water usage.

It is a further object, feature, and/or advantage of the present disclosure to provide larger CAP arrays that produce reactive species that sanitize surfaces and prevent pathogenic biofilm accumulation. According to some aspects of the present disclosure, the CAP treatment will rapidly inactivate pathogens on higher volumes of agricultural and food products or medical surfaces. According to some aspects of the present disclosure, the CAP array devices recirculate reactive plasma species to recycle working gas and enhance antimicrobial effectiveness.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Disclosed herein are Cold atmospheric pressure plasma (CAP) array systems comprising: a) a CAP array comprising a discharge element comprising two discharge plates each comprising a conducting electrode covered with a dielectric material, and a dielectric spacer configured to define the distance between discharge plates; b) a gas flow inlet configured to flow a working gas through the CAP array; c) a showerhead configured between the gas flow inlet and the CAP array; and d) a conducting rod at each end of the CAP array configured to establish electrical connection to the conducting electrode of each discharge plate; wherein the discharge plates are stacked together and electrically connected in parallel through the conducting rods, and wherein one conducting rod is in electrical connection with a power supply and the other is in electrical connection with a power supply or to ground.

In some embodiments, the cold atmospheric pressure plasma array systems comprise more than two, more than eight, more than 20, more than 40, or about 43 discharge elements, wherein each discharge plate, excluding two end plates, comprise an electrode on each side of said plate, and wherein the discharge elements are stacked together and electrically connected in parallel through the conducting rods. In some embodiments, the discharge plates further comprise a ballast resistor or a capacitor in electrical connection with the conducting electrode, wherein the ballast resistor is connected to the conducting electrode with silver paint and/or solder, and wherein the ballast resistors of two adjacent plates are on opposite ends of the plates, connected to electrodes at different potentials. In some embodiments, the showerhead comprises round holes that are not uniform in size or distribution, wherein the showerhead comprises smaller holes in the center half of the showerhead.

In some embodiments, the cold atmospheric pressure plasma array systems further comprise an enclosure configured to house the CAP array, the showerhead, the gas inlet, wherein the gas flow inlet comprises a fan and the working gas comprises air.

In some embodiments, the system further comprises a capture system adjacent to at least one side of the CAP array configured to capture gas effluent, wherein the capture system comprises a negative pressure region, and wherein the capture system comprises an exhaust system and/or filtration configured to remove pathogenic detritus from the system. In some embodiments, the capture system comprises a gas return path configured to recirculate the effluent back to the gas inlet.

Disclosed herein are methods of treating a surface comprising applying voltage to and flowing a working gas through the cold atmospheric pressure plasma array system of claim 1, and exposing a surface to a plasma generated by the cold atmospheric pressure plasma array system, wherein the plasma comprises reactive oxygen and nitrogen species and wherein the working gas comprises argon, nitrogen, oxygen, ambient air, or combinations thereof, and wherein the method results in colony forming unit (CFU) reduction of at least about 50%, at least about 60%, at least about 70%, at least about 80%, at least about 90%, or about 100%.

In some embodiments, the surface is from about 5 mm to about 100 mm from the CAP array, wherein the surface is exposed to the plasma for about 150 seconds to about 5 minutes, wherein the flow rate of the working gas is from about 1 LPM to about 300 LPM, and/or wherein the voltage is from about 0.9 kVrms to about 5.0 kV. In some embodiments, the working gas is air and the and the voltage is from about 2 kV to about 3 kV.

In some embodiments, the method comprises more than one cold atmospheric pressure plasma array system.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows an exploded view of a system with 43 discharge elements with 1.1 mm discharge gaps. The discharge elements are LTCC with electrodes buried 100 μm below a dielectric. Ballast resistors are connected to each electrode. Copper rods connect the electrodes through a silver paint. Gas enters the back enclosure and is dispersed through the showerhead for gas flow uniformity.

FIG. 2 shows the assembled structure of FIG. 1.

FIG. 3 is a photograph of a 10 cm×10 cm full array device with an active plasma.

FIG. 4 shows a four-element plasma array assembly. LTCC spacer plates separate high voltage electrode and ground plates with embedded ballast resistors.

FIG. 5 is a photograph of a 10 cm×10 cm CAP array with attached fan to provide air gas source.

FIG. 6 shows an illustration of a single linear CAP device in use.

FIG. 7 shows two drawings of an enclosure designed to capture gas from the plasma array. The capture region uses negative pressure to pull the effluent into the tubes.

FIG. 8 shows an exemplary housing for the device array and a capture system to safely contain and filter ozone before releasing purified air into the surroundings. A sealing rim ensures no ozone leakage while a fan directs the gas into funnels that channel collected ozone through a carbon filter neutralizing harmful compounds before releasing clean air back into the environment.

FIG. 9 is a photograph of a robot with a device array and capture system attached to an arm for autonomous sterilization.

FIG. 10 is a side view of the robot and attached array shown in FIG. 8.

FIG. 11 is a drawing of a conveyer belt system wherein CAP arrays are placed above and underneath a conveyer belt. The CAP arrays above to sanitize an object in need of sterilization while the CAP arrays underneath sanitize the convey belt material.

FIG. 12 shows the representation of a 3-element plasma device with electrical components.

FIG. 13 is a photograph of generations of the discharge plate showing changes in via type, connections, and positions.

FIG. 14 is an illustration of the CAP device setup as used in the Examples.

FIG. 15 is an exploded view of an array arrangement wherein plates are separated by 1.1 mm spacers and the ballast resistor connection and the parts are connected electrically with silver tape.

FIG. 16 is a top-down view of an assembly wherein copper rods are utilized for electrical connection.

FIG. 17 are photographs of a 9-element array tested with four different showerheads.

FIG. 18 is a photograph of a 9-element array device with a larger power supply.

FIG. 19 shows two photographs of showerhead designs.

FIG. 20 is graph of current versus voltage of an array of 23 elements with 10 kΩ resistor.

FIG. 21 is a graph of current versus the number of elements in an array with a silver tape connection and an array connection with copper rods.

FIG. 22 is a graph of the capacitance of each layer in each array.

FIG. 23 shows the device with ceramic paste and Kapton tape and the resulting plasma bright spots.

FIG. 24 is an I-V curve of a 43-element array with version 4.0 discharge plates and 20 kΩ ballast resisters on both sides.

FIG. 25 is a graph of CFU per treatment.

FIG. 26 is a graph of CFU per treatment for a biofilm grown on laminate.

FIG. 27 is an illustration of the embodiment in FIG. 11.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable including but not limited to mass, volume, temperature, voltage, capacitance, current, distance, time, frequency, flow rate, and resistance. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “configured” describes structure capable of performing a task or adopting a particular configuration. The term “configured” can be used interchangeably with other similar phrases, such as constructed, arranged, adapted, manufactured, and the like.

Terms characterizing sequential order (e.g., first, second, third), a position, and/or an orientation are not limiting and are only referenced according to the views presented.

When an element is referred to as being “connected,” “coupled,” “mated,” “attached,” “fixed,” etc. to another element, the element can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.). Similarly, terms such as (i) “communicatively connected” or (ii) “fluidly connected” include (i) all variations of information exchange and routing between two electronic devices, including intermediary devices, networks, etc., connected wirelessly or not and (ii) all variations of fluid exchange and routing between two fluidic bodies, including intermediary fluid paths, flows, etc., connected indirectly or not.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components or ingredients, but only if the additional steps, components or ingredients do not materially alter the basic and novel characteristics of the claimed methods, systems, apparatuses, and compositions.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

As used herein, “health care surface” or “medical surface” refers to a surface of an instrument, a device, a cart, a cage, furniture, a structure, a building, or the like that is employed as part of a health care or medical activity. Examples of such surfaces include, but are not limited to, surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, and clinical diagnosis rooms. These surfaces can be those typified as “hard surfaces” (such as walls, floors, bed-pans, etc.), or fabric surfaces, e.g., knit, woven, and non-woven surfaces (such as surgical garments, draperies, bed linens, bandages, etc.), or patient-care equipment (such as respirators, diagnostic equipment, shunts, body scopes, wheel chairs, beds, etc.), or surgical and diagnostic equipment. Health care surfaces include articles and surfaces employed in animal health care.

As used herein, the term “instrument” refers to the various medical or dental instruments or devices that can benefit from the method as described herein. As used herein, the phrases “medical instrument,” “dental instrument,” “medical device,” “dental device,” “medical equipment,” or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning in a composition of the present disclosure. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressers, trocars, dilators, cages, glassware, tubing, catheters, cannulas, plugs, stents, scopes (e.g., endoscopes, stethoscopes, and arthroscopes) and related equipment, and the like, or combinations thereof.

As used herein, the phrase “food processing surface” refers to a surface of a tool, a machine, equipment, a structure, a building, or the like that is employed as part of a food or beverage processing, preparation, or storage activity. Examples of food processing surfaces include, but are not limited to, surfaces of food processing or preparation equipment (e.g., boiling, fermenting, slicing, canning, or transport equipment, including flumes), of food processing wares (e.g., utensils, dishware, wash ware, and bar glasses), and of floors, walls, or fixtures of structures in which food processing occurs.

As used herein, the phrase “food product” or “food” includes any food substance that might require treatment and that is edible with or without further preparation. Food products include meat (e.g. red meat and pork), seafood, poultry, produce (e.g., fruits and vegetables), eggs, living eggs, egg products, ready to eat food, wheat, seeds, roots, tubers, leafs, stems, corns, flowers, sprouts, seasonings, or a combination thereof.

As used herein, the term “microorganism” refers to any noncellular or unicellular (including colonial) organism. Microorganisms include all prokaryotes. Microorganisms include bacteria, spores, lichens, fungi, protozoa, virinos, viroids, viruses, phages, and algae. As used herein, the term “microbe” is synonymous with microorganism.

As used herein, the phrase “plant” or “plant product” includes any plant substance or plant-derived substance. Plant products include, but are not limited to, seeds, nuts, nut meats, cut flowers, plants or crops grown or stored in a greenhouse, house plants, and the like.

As used herein the term “polymer” refers to a molecular complex comprised of a more than ten monomeric units and generally includes, but is not limited to, homopolymers, copolymers, such as for example, block, graft, random and alternating copolymers, terpolymers, and higher “x”mers, further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

As used herein, the term “treat”, “treated”, “treatment”, “treating” or like terms when used refers to decreasing or reducing the amount of a microorganism on a surface, or inhibiting at least one function of the microorganism, or disrupting a biofilm, and/or inhibiting a biofilm's formation. In some embodiments, “treat,” “treated,” “treatment” or “treating” refers to a therapeutic or prophylactic treatment that disrupts a biofilm or part thereof. As used herein “sanitizing” and “disinfecting” refer to decreasing, reducing, or inhibiting at least one function of a microorganism. In some instances “sanitizing” or “sanitation” or “disinfecting” or “disinfection” refers to reducing the number of a microorganism on a surface to a safe level. “Treating” also includes inactivating bacteria, viruses, and protozoa within biofilms. As used herein, “inactivating” or “inactivation” includes a treatment that renders a microbial biofilm or microorganism ineffective, or a treatment that disrupts a biofilm's structure thereby preventing said biofilm from functioning as a protective barrier for bacteria or other microorganisms. “inactivating” or “inactivation” encompasses the termination of bacterial, biofilm, or microorganism growth.

The term “virus”, as used herein refers to a type of microorganism that can include both pathogenic and non-pathogenic viruses. Pathogenic viruses can be classified into two general types with respect to the viral structure: enveloped viruses and non-enveloped viruses. Some well-known enveloped viruses include herpes virus, influenza virus; paramyxovirus, respiratory syncytial virus, corona virus, HIV, hepatitis B virus, hepatitis C virus and SARS-COV virus. Non-enveloped viruses, sometimes referred to as “naked” viruses, include the families Picornaviridae, Reoviridae, Caliciviridae, Adenoviridae and Parvoviridae. Members of these families include rhinovirus, poliovirus, adenovirus, hepatitis A virus, norovirus, papillomavirus, and rotavirus.

As used herein a “biofilm” is a community of microorganisms that adhere to each other and to a surface.

### Larger Scale CAP Array Devices

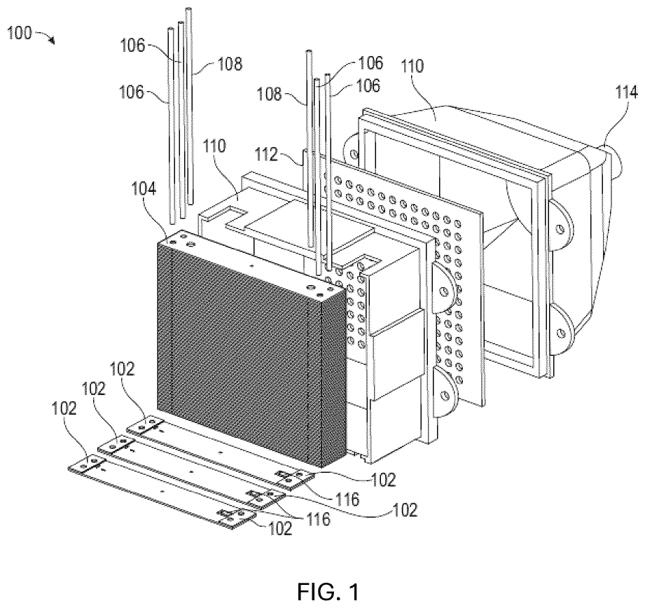

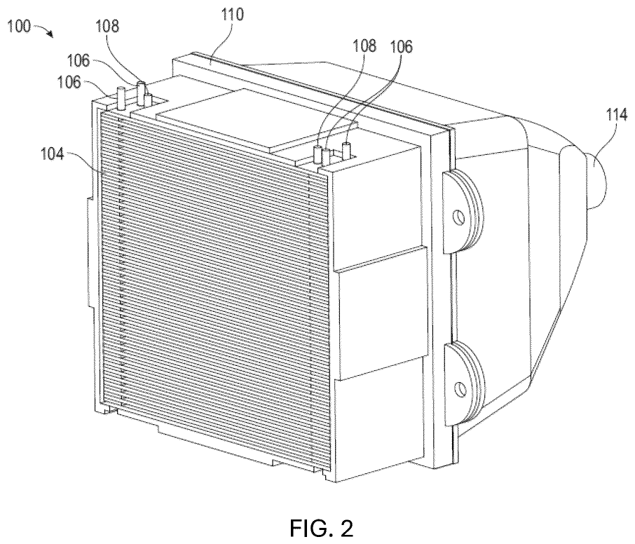

Exemplary larger scale CAP array devices 100 as disclosed herein are shown in FIG. 1 and FIG. 2. An exploded view diagram of an exemplary CAP array device 100 is shown in FIG. 1. This embodiment comprises 43 discharge elements 104. The device comprises 44 plates 104, two of which are end plates, in a parallel electrical configuration. Electrodes on both sides of the discharge plates are electrically shorted to each other. The discharge gap is fixed by way of Delrin spacers 102 on each side of the plate. This embodiment includes a total of six rods. Four insulating rods 106 at the edge on both sides of the device are assembly rods. These rods hold the assembled device together. Two inner conducting rods 108 establish electrical connection among the discharge plates. Conducting rods 108 on both sides of the device are connected to a power supply. The device is configured for gas to be introduced through a gas inlet 114 at the back of the enclosure. A showerhead 112 is placed between the device and the gas inlet for uniform gas distribution. The large scale CAP array is enclosed in a housing 110.

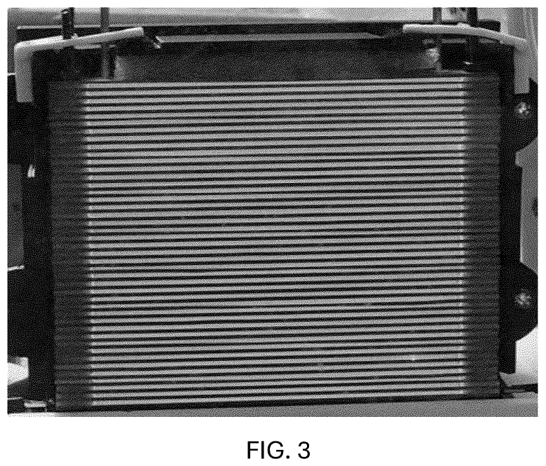

The assembled exemplary device 100 is shown in FIG. 2. FIG. 3 shows a photograph of the exemplary array structure in use.

Disclosed herein are CAP array devices comprising stacked linear discharge elements, also referend herein as “discharge plates,” or “plates” wherein the conducting electrodes are covered with dielectric material to prevent deterioration. Plasma is generated through electron impact ionization. This is known as Dielectric Barrier Discharges (DBD). The elements are stacked together and electrically connected in parallel through conducting rods. The CAP devices generate reactive gas species and deliver them onto surfaces to effectively inactivate harmful bacteria.

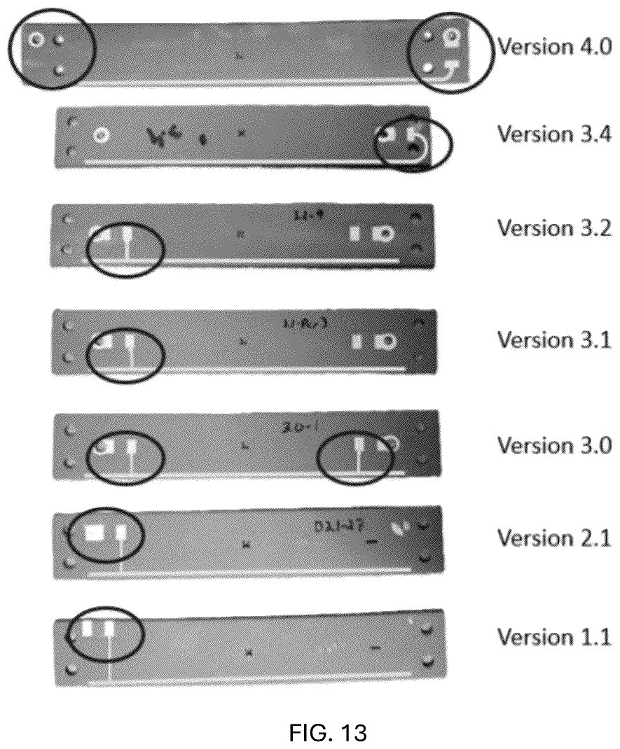

The discharge plate comprises a conducting electrode covered with dielectric material. In an embodiment, each discharge plate comprises a conducting electrode on each side of the discharge plate. The conducting electrode can be any electrode as known in the art. In a preferred embodiment, the electrode comprises silver, for example silver tape or paste. In an embodiment, the electrode is positioned within the discharge plate to be closer to the treatment surface than the gas flow introduction. In an embodiment, the electrode is positioned across the length of the discharge plate, or substantially the length of the discharge plate, or the length of the discharge plate excluding the spacer. Exemplary embodiments of the electrode placement on the discharge plate are shown in FIG. 13. In an embodiment, the electrode is a substantially straight line across the bottom portion of the discharge plate and then curves to make connection with the ballast resistor. This embodiment is shown in Version 3.4 and Version 4.0 in FIG. 13. In an embodiment, the shape of the electrode does not comprise any right angles. In an embodiment, the electrode is covered with a dielectric material. In a preferred embodiment, the electrode is covered with a ceramic material. In an embodiment, the electrode is covered with about 50 μm to about 200 μm of dielectric.

The discharge plate can comprise any dielectric material as known in the art. In a preferred embodiment, the discharge plate comprises ceramic. In a preferred embodiment the discharge plate comprises Low Temperature Co-Fired Ceramic (LTCC) material. LTCC is versatile and adaptable to different designs and needs. A versatile and adaptable ceramic such as LTCC allows for construction of various shapes by layering multiple layers of LTCC before curing with heat. Therefore, LTCC is relatively easy to fabricate more complex structures. The discharge plate can be any size or shape as known in the art. For example, a square, a rectangle, an oval, and the like. In a preferred embodiment, the discharge plate is a rectangular shape about 12 cm long, about 2 cm wide, and about 1.2 mm thick.

In an embodiment, the CAP array 100 comprises at least two discharge plates 104. In an embodiment, the CAP array comprises at least 8 discharge plates 104. In an embodiment, the CAP array comprises 10 or more discharge plates 104. In an embodiment, the CAP array comprises 20 or more discharge plates 104. In an embodiment, the CAP array comprises 40 or more discharge plates 104. In a preferred embodiment, the CAP array comprises 44 discharge plates 104 (for 43 discharge elements) for a 10 cm×10 cm plasma.

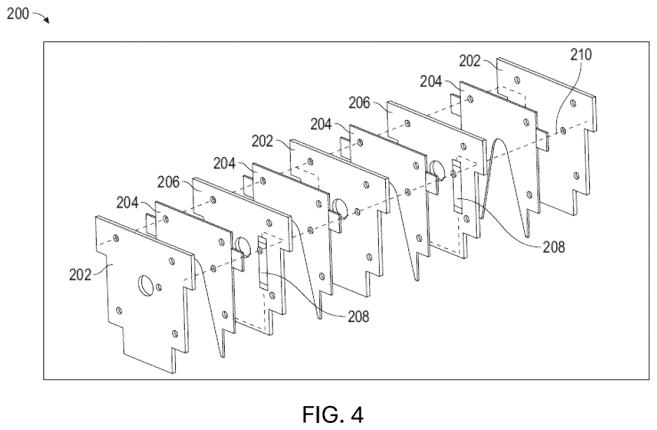

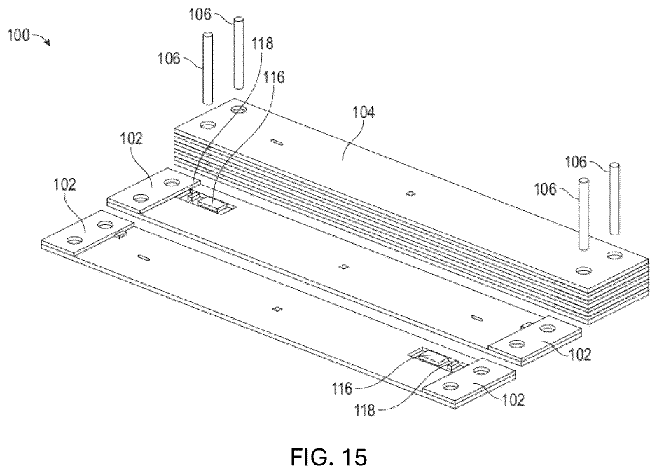

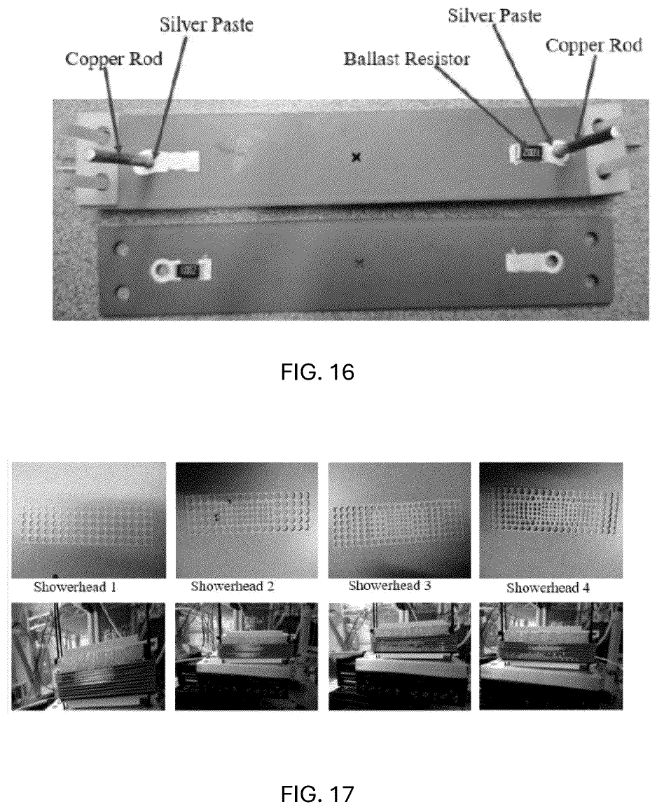

In an embodiment, the CAP arrays as described herein further comprise a spacer configured to define the distance between discharge plates. The spacer can be any dielectric material, or insulator, configured to handle the device temperature. In an embodiment, the spacer is a spacer plate 204 as shown in FIG. 4 and illustrated in FIG. 6. In an embodiment, the spacer is a Delrin spacer 102. Exemplary Delrin spacers are shown in FIG. 1, FIG. 15 and FIG. 16. The spacer can be any dielectric or insulating material as known in the art. In an embodiment, the spacer is configured such that the space between the discharge plates is about 0.5 mm to about 2 mm, or between about 0.7 mm to about 1.1 mm. In a preferred embodiment, the spacer is configured such that the space between the discharge plates is less than about 1.5 mm. In a preferred embodiment, the spacer is configured such that the space between discharge plates is 1.1 mm or less.

In an embodiment, the CAP arrays as described herein further comprise a ballast resistor in electrical connection with the electrode. Without being limited to a particular theory, the incorporation of a ballast resistance serves to minimize the variation in discharge current across individual channels, preventing the intense discharge of one channel before others have started. This proves advantageous in the context of parallel configurations where it facilitates more precise control over the discharge process and concurrently diminishes the potential for intense bright spots or filaments. Overall, the inclusion of ballast resistance emerges as a valuable technique for optimizing plasma uniformity and controlling discharge dynamics. In an embodiment, the discharge plates comprise a capacitor in electrical connection to serve as a ballast impedance with the conducting electrode. Beneficially, the inclusion of a capacitor reduces heat dissipation, bringing similar benefits to the CAP array.

In an embodiment, the ballast resistor 208 is embedded within a plate as shown in FIG. 4. In an embodiment, the ballast resistor 116 is incorporated into the discharge plate 104 as shown in FIG. 1, FIG. 15 and FIG. 16. In an embodiment, the ballast resistor is a 2 W ballast resistor. In an embodiment, the CAP array 100 comprises ballast resistors 116 of two adjacent plates on opposite ends of the plates. This embodiment is shown in FIG. 1, FIG. 15 and FIG. 16. In this embodiment, alternating ballast resistor 116 are connected to electrodes at different potentials. The ballast resistor 116 can be in electrical connection with the electrode by any means as known in the art. In an embodiment, the ballast resistor is electrically connected to the electrode with solder. In a preferred embodiment, the ballast resistor is electrically connected to the electrode with silver paint. In an embodiment, the ballast resistor comprises a resistance of from about 10 kΩ to about 20 kΩ. In an embodiment, the ballast resistance is about 20 kΩ, or at least about 20 kΩ. In an embodiment, the resistance is less than about 10 kΩ, or about 5 kΩ. In an embodiment, the resistance is higher than about 20 kΩ, or about 40 kΩ. In an embodiment, the discharge plates comprise a capacitor with the same impedance at the operating frequency.

In an embodiment, the electrode within each discharge plate within the CAP array 100 are in electrical connection through a conducting rod 108 that extends through the end of every discharge plate in the array at each end. In a preferred embodiment, the conducting rod 108 comprises copper. In a preferred embodiment, the array 100 further comprises silver paste or tape 118 to ensure connection of the copper rod 108 to the discharge plate 104 electrode. This embodiment is shown in FIG. 1, FIG. 15, and FIG. 16. In an embodiment, the discharge plate is configured such that the copper rod is not in the active region of the discharge plate. This embodiment is shown in version 4.0 of the discharge plate in FIG. 13. In an embodiment, the conducting rod is connected to the electrode with solder.

In an embodiment, the conducting rod on one side of the CAP array is configured for electrical connection to ground and the other is configured for electrical connection to a voltage supply.

The CAP array can be held together by any means as known in the art. In an embodiment, the CAP array comprises a nonconducting threaded assembly rod at each end of the array configured to hold the assembled device together. In an embodiment, bolts are attached to the threaded rods at the top and bottom of the device. In an embodiment, the CAP array 100 is held together by two insulating assembly rods 106 at each end of the discharge plates 104. This embodiment is illustrated in FIG. 1, FIGS. 15 and 16. In an embodiment, the assembly rods are insulating.

In an embodiment, the CAP array device comprises a showerhead for uniform gas flow. The showerhead is configured between the gas flow inlet and the CAP array. The showerhead can be made of any insulating material as known in the art. In an embodiment, the showerhead comprises round holes as shown in FIG. 17 and FIG. 19. In an embodiment, the showerhead comprises round holes that are not uniform in size or distribution. In an embodiment, the showerhead comprises smaller holes in the center half of the showerhead. In an embodiment, the showerhead comprises holes with about 4 mm diameter in the outer ⅓ to outer ⅔ of the showerhead surface and holes with a diameter of about 2 mm in the inner ⅓ to inner ⅔ of the surface area of the showerhead.

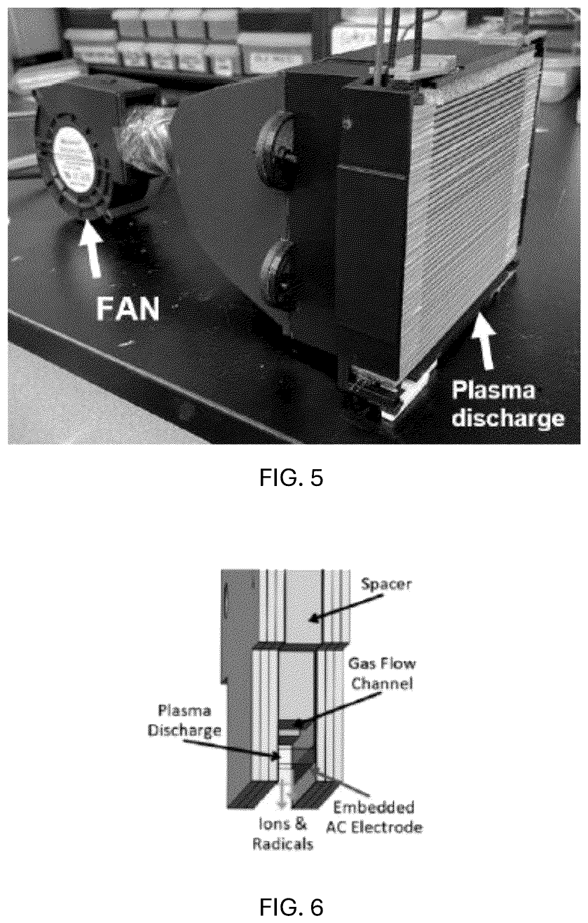

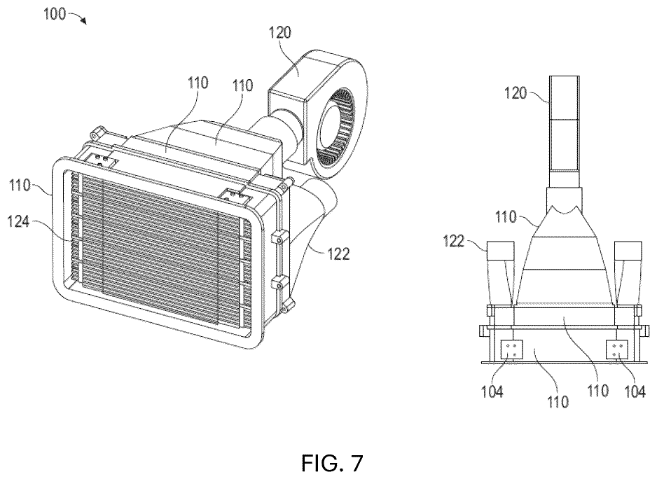

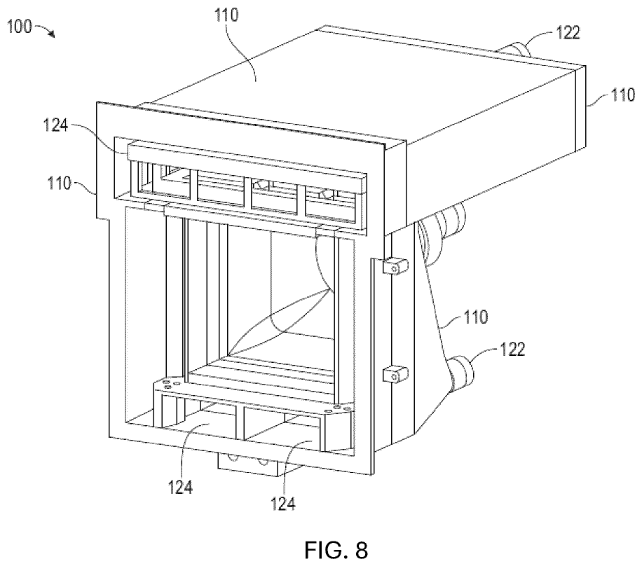

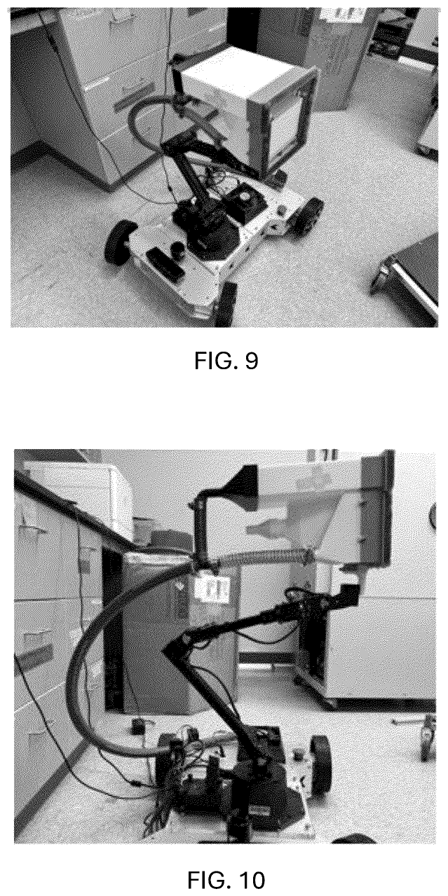

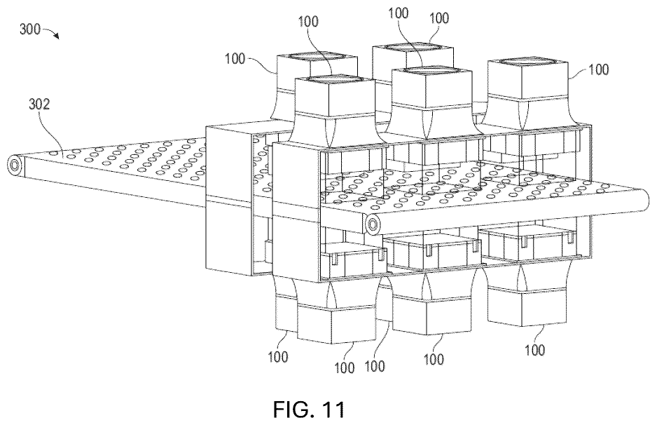

In an embodiment, the CAP array device comprises an enclosure 110 configured to house the CAP array, the showerhead, and in some embodiments the gas inlet. The enclosure can be made of any insulating material as known in the art. The enclosure can be of any size and shape configured to enclose the CAP array device elements. In an embodiment, the enclosure is 3D printed. Exemplary enclosures are shown in FIG. 1, FIG. 2, FIG. 5, FIG. 7, FIG. 8, FIG. 9, FIG. 10, and FIG. 11. The enclosure can also be covered with a conductor to reduce noise generation beyond the enclosure.

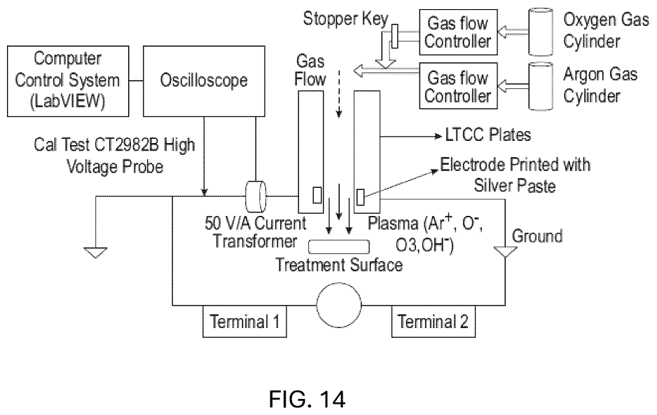

In an embodiment, the CAP array device comprises a gas flow inlet 114 configured to flow gas through the CAP array. This is illustrated in FIG. 1, FIG. 2 and FIG. 14. In an embodiment, the gas flow inlet is connected to gas flow controller, gas source, and the like and combinations thereof. In an embodiment, the working gas comprises oxygen, nitrogen, argon, or combinations thereof. Water may also be introduced into the gas. The gas flow inlet can be configured to flow the working gas through the array by any means as known in the art. In an embodiment, the gas flow inlet comprises a fan 120 and the working gas comprises room air. This embodiment is shown in FIG. 5 and in FIG. 7.

In an embodiment, the CAP array structure comprises a capture system 124 configured to capture gas effluent after treatment. In an embodiment, the capture system comprises a negative pressure region to pull, for example, reactive oxygen and nitrogen species (RONS) that were used to inactivate pathogens. The negative pressure region can be devised by any means as known in the art, for example a fan to provide suction. In an embodiment, the fan providing gas flow also provides suction for the capture system. In an embodiment. the capture system comprises an exhaust system. In an embodiment, the capture system comprises a filter. In embodiments, the exhaust system and/or filtration removes pathogenic detritus from the system. In an embodiment, the exhaust and/or filtration system removes gas species such as ozone and/or hydrogen peroxide. In an embodiment, the capture system comprises a material that contacts at least a portion of the treatment surface. In an embodiment, the capture system comprises a material that contacts at least a portion of the treatment surface forming a closed system to prevent gas species release. In an embodiment, the material is fibrous. In an embodiment, the capture system is an open system, wherein the capture system does not fully enclose the treatment surface.

Exemplary capture systems are shown in FIG. 7 and FIG. 8.

In an embodiment, the capture system comprises a gas return path 122 configured to recirculate the effluent comprising RONS back to the gas inlet. In an embodiment, the gas capture system 124 is adjacent to at least one side of the CAP array. In an embodiment, the gas capture system 124 surrounds the CAP array on all sides.

In an embodiment, the enclosure 110 comprises the capture system 124. In an embodiment, the capture system comprises the enclosure.

In an embodiment, the CAP array device comprises a power source with each terminal of the source connected to one of the conducting rods of the array. In this embodiment, the power source comprises an A/C source with its frequency controlled as desired for best results. In this embodiment, one of the terminals may be established as ground terminal. The voltage source can be any as known in the art.

In an embodiment, the CAP array device comprises a current transformer configured for electrical connection with the power supply and a conducting rod.

In an embodiment, the CAP array device comprises a cooling device and/or cooling system. In an embodiment, the cooling system comprises a polymer configured at the edges of the CAP array elements to pull heat away from the array and ballast resistors. In an embodiment, the cooling system comprises spacers with high thermal conductivity. In an embodiment, the cooling system comprises copper plates configured to trap polymer against the array, and these plates will extend back behind the array to convective cooling fins or a conductive water coolant system. In an embodiment, the cooling device and/or cooling system is within the enclosure of the device.

In an embodiment, the CAP array device comprises a plasma driver. In an embodiment, the driver is configured to allow operation at high voltages (~2500 Vrms) and current (~200 mA) using air as the working gas. In an embodiment, the plasma driver is configured to allow programmed control to autotune the resonant frequency after plasma initiation. In an embodiment, the driver comprises a controller, start and/or shut-down software, current and/or temperature sensors, shut down circuitry, and combinations thereof.

In an embodiment, the CAP array device comprises any measurement system and/or detection system as known in the art. For example, but not limited to, this includes any device and/or system to measure, monitor, and/or control temperature, voltage, current, gas flow, effluent characteristics, working gas level, and the like. In an embodiment, the CAP array device comprises RONS detection. In an embodiment, the CAP array device comprises an ozone detector. In an embodiment, the CAP array device comprises a peroxide detector. In an embodiment, the production of reactive hydrogen peroxide (H₂O₂) is measured with an Amplex Red® Hydrogen Peroxide/Peroxidase kit (ThermoFisher) that detects fluorescent resorufin (ex/em 571 nm/585 nm) using a 96-well plate reader. In this embodiment, the plasma device is discharged into a petri dish containing 10 mL of DI water placed 1-4 cm below the device. The liquid is sampled periodically and the AmplexRed kit used to determine dissolved peroxide. A standard curve of known concentrations of peroxide is used to assign test sample concentrations. In an embodiment, the CAP array device comprises a system to detect and/or monitor hydroxyl radicals. In an embodiment, hydroxyl radicals (·OH) are detected by discharging the plasma into 10 mL terephthalate (TPA) solution and measuring the formation of fluorescent hydroxyterephthalate (excitation/emission: 315 nm/425 nm). A standard curve for hydroxyl radicals is generated using 10 μM Cu2+ ion and 0-30 mM H₂O₂. In an embodiment, the CAP array device comprises a device or system to detect and/or monitor superoxide. In an embodiment, superoxide anion/radical (O²⁻/·O²⁻) is measured by discharging the plasma into a petri dish containing a 10 mL volume of DI water. Dissolved superoxide is measured using a Superoxide Anion Assay kit (Sigma-Aldrich) that detects the chemiluminescence evolved when superoxide reacts with luminol. Known concentrations of xanthine and xanthine oxidase provide a standard curve for the assay.

In an embodiment, the CAP array device is placed on any surface as suitable for use, stationary or otherwise. In an embodiment, the CAP array device as described herein is mounted on a robot or robot arm or movable structure. This embodiment can be used for example, but not limited to, hands free surface sanitizing. Exemplary embodiments are shown in FIG. 9 and FIG. 10.

In an embodiment, more than one or several CAP array devices as described herein are utilized together for form large surface sanitation. An exemplary system 300 comprising five CAP array devices 100 above and below a conveyor belt 302 is shown in FIG. 11. In this exemplary embodiment, the five CAP array devices 100 on the top of the conveyor belt treat a surface moving down said conveyor belt 302. The treated surface can be anything as known in the art-food, food preparation surfaces, seeds, medical equipment, and the like. The conveyor belt 302 itself is then sanitized by the five CAP array devices on the underside of the belt. FIG. 27 is an illustration of this embodiment.

### Gas Capture System

Described herein is a gas capture system for a CAP array, not limited to the CAP arrays as described herein.

In an embodiment, the CAP array gas capture system comprises a CAP plasma forming structure and a capture system configured to capture gas effluent after treatment. In an embodiment, the capture system comprises a negative pressure region to pull any effluent, but particularly reactive oxygen and nitrogen species (RONS) that were used in the plasma. The negative pressure region can be devised by any means as known in the art, for example a fan to provide suction. In an embodiment, the fan providing gas flow to a CAP plasma forming structure also provides suction for the capture system. In an embodiment, the capture system comprises a material that contacts at least a portion of a treatment surface. In an embodiment, the capture system comprises a material that contacts at least a portion of the treatment surface forming a closed system to prevent gas species release. In an embodiment, the material is fibrous.

In an embodiment, the capture system comprises an exhaust system. In an embodiment, the capture system comprises a filter. In embodiments, the exhaust system and/or filtration removes pathogenic detritus from the system. In embodiments, the exhaust system and/or filtration removes ozone and/or hydrogen peroxide and/or other reactive species from the system.

In an embodiment, the CAP array gas capture system comprises a gas return path configured to recirculate the effluent, in some embodiments comprising RONS, back to the gas inlet.

In an embodiment, the CAP array gas capture system comprises an enclosure wherein the enclosure is configured to house the CAP plasma forming structure and the capture system and the optional gas return. The enclosure can be made of any insulating material as known in the art. The enclosure can be of any size and shape.

### Methods of Use

Described herein are methods of using any of the CAP array devices as described herein to treat a surface. The effectiveness of cold atmospheric pressure plasma in sanitizing and disinfecting surfaces is known. Various mechanisms have contributed to this sanitization process, such as UV photons, interactions with electric fields, thermal reactions, and the reaction with reactive oxygen and nitrogen species (ROS and RNS) produced by the plasma. These reactive species such as, O²⁻, O₃, OH—, H₂O₂, NO, and N₂O are formed by the plasma collision, and these reactive species cause both intracellular and extracellular effects that deactivate harmful biological species by interacting with the cellular membrane, proteins, fatty acids, and nucleic acids. Additionally, the inactivation process may include cell membrane damage due to ion impacts, DNA mutations, and programmed cell death (apoptosis).

A basic one element plasma device is essentially a capacitor, where high potential is applied between two parallel conductors to ionize the gas present between them. The same concept is applied here; however, in this case, the electrodes are covered by LTCC dielectric, and the applied potential is high enough to generate a dielectric barrier discharges.

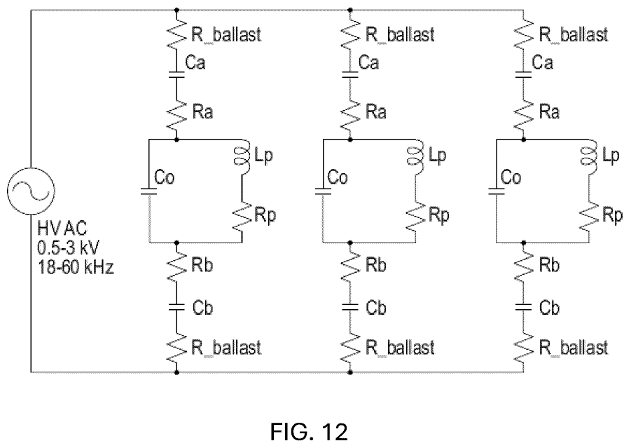

To create the larger CAP array devices and described herein, multiple discharge plates are stacked electrically in parallel. An equivalent circuit of a 3-element plasma device is presented in FIG. 12. Plasma can be represented by a series combination of plasma resistance (Rp), plasma inductance (Lp), and sheath capacitance (Ca and Cb). Ra and Rb are the resistances of the two sides of the sheath. Co is the array capacitance between two electrodes. Two ballast resistors (R_ballast) are connected with each element to improve plasma uniformity. Generally, one ballast resistor connected to one of the conducting rods is enough for small devices. For larger devices as described herein, more than one, or at least two ballast resistors connected to both conducting rods are used to get uniform plasma throughout the device.

As known in the art. Important factors for device optimization include the gas used, the distance between the plasma device and the treated surface, the distance between electrodes, the applied voltage, and the discharge gap.

Described herein are methods of treating a surface comprising exposing a surface to the plasma of any one of the CAP array devices as described herein. In an embodiment, the method results in reduced amounts of microorganisms or biofilm on said surface and/or inactivation of at least a portion of microorganisms and/or biofilm on said surface.

In an embodiment, the method comprises flowing a working gas into the CAP array device as described herein, applying a voltage to the CAP array device as described herein, and exposing a surface to the plasma. In an embodiment, the working gas comprises argon, nitrogen, oxygen, ambient air, or combinations thereof. In an embodiment, the gas source comprises the molecular forms of the gas (e.g. N₂, O₂, and Ar). In an embodiment, the flow rate of the working gas is from about 1 LPM to about 300 LPM. In a preferred embodiment, the working gas comprises ambient air.

In an embodiment, the method results in colony forming unit (CFU) reduction of at least about 50%, at least about 60%, at least about 70%, at least about 80%, at least about 90%, at least about 90%, or about 100%.

In an embodiment, the voltage is from about 0.5 kV to about 5.0 kV, with currents ranging from about 100 mA to about 300 mA. In an embodiment, the working gas comprises argon and oxygen and the voltage is from about 1 kV to about 2 kV, or from about 1.15 kV to about 1.5 kV with currents ranging from about 100 mA to about 200 mA or from about 125 mA to about 170 mA. In an embodiment, the working gas comprises air, and the voltage is from about 2 kV to about 3 kV, or from about 1 kV to about 5 kV, and the current ranges from about 150 mA to about 250 mA.

In an embodiment, the method comprises a treatment time of from about several seconds to tens of minutes. In an embodiment, the method comprises a treatment time of from about 150 seconds to about 5 minutes. In a preferred embodiments, the surface is exposed to the plasma for less than about 60 seconds. In embodiments comprising a cooling system and/or driver system, the treatment time can be several minutes or longer.

In an embodiment, the method comprises placing the surface in need of treatment at a distance of from about 5 mm to about 100 mm from the CAP array. In an embodiment, the method comprises placing the surface in need of treatment at a distance of from about 15 mm to about 30 mm from the CAP array.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

For the Examples presented herein, the following procedures were followed unless otherwise indicated.

An experimental setup (showing one element) of a CAP device utilized herein is presented in FIG. 14. Shown are LTCC discharge plates with silver paste printed electrodes. Gas flow controllers were used to control gas flow rate to the discharge gaps. One side of the CAP device was connected with a High Voltage AC power supply and the other side was connected to ground. A Cal Test CT2982B High Voltage (HV) Probe and 50 V/A current transformer were connected between HV AC power supply and CAP device to measure AC voltage and current to the device. The high voltage probe and current transformers were connected to an oscilloscope, and the output was read by Lab VIEW, a computer control system application, to process and display the parameters on a computer screen. Between about 0-10 kV HV AC at 18-60 kHz frequency was applied to operate the device.

The entire assembly was enclosed in a test box to provide exhaust gas capabilities and HV interlocks. The CAP device, the current transformer and the high voltage probe were placed inside the test box. A 20-266 LPM gas flow controller, connected with a gas tube, was attached to the box. A negative pressure vacuum system outlet was connected to the box to exhaust gas species such as ozone. The test box was closed to engage the safety interlock, and the vacuum exhaust was activated before testing began.

The device was assembled by stacking discharge plates together. The array plate design was modified multiple times to resolve various problems as described in these Examples. Each discharge plate had two electrodes, one on each side, and these electrodes were connected together through vias. On each plate there was a surface mount resistor connected using silver paste. Four nylon bolts were placed in screw holes to hold the assembly together, and laser cut Delrin spacers established the discharge gaps. Each discharge plate comprised two vias, one on each end of the plate. One via was attached to the resistor pad. Folded adhesive silver tape sheets were used to make electrical connections between two discharge plates through vias. In the examples, the plates are approximately about 12 cm long, about 2 cm wide, and about 1.2 mm thick.

I-V sweeps were obtained by operating the device in different configurations and conditions to analyze the device characteristics. For the I-V sweep tests, the device was assembled, placed in an appropriate enclosure, connected to the HV AC power supply and ground sides with HV compatible cables, and the appropriate gas flow rate was injected to setup the experiment. The I-V sweeps were taken by increasing the voltage gradually from zero to the highest possible voltage and taking note of the corresponding current measured by the current transformer. Because the device was assembled by stacking multiple discharge plates together with a 0.5-2 mm spacer in between, it forms an arrangement of multiple parallel capacitors. The capacitance of the device and/or the plates was measured with a handheld U1733C LCR meter during assembly at a frequency of 10 KHz.

### Example 1—Demonstrating Uniform Plasma

In this Example, a 10 kΩ ballast resistor and a 3-element array with version 2.1 plates according to FIG. 13 was tested. Only the center portion of the array created plasma due to nonuniform gas flow. Because the enclosure had a gas inlet at the center, only the middle part of the device could get enough argon gas to create plasma, leaving the rest of the device inactive. More experiments with 3, 5, 7, and 9-element arrays were performed. However, all had the same nonuniformity issues. I-V sweeps were taken for the various arrays, showing different voltages required to turn on the different sizes of arrays. The 3-element array required around 850 Vᵣₘₛ to turn on, whereas the 9-element array required around 1.8 kVᵣₘₛ. Before plasma turn on, displacement current flows through the devices. Conduction current occurs between plates once the plasma starts. Since the plasma is not uniform, the conduction current was not linear resulting in the discontinuities in I-V sweeps.

For uniform plasma, a 9-element CAP array device was tested with four different showerhead designs as shown in FIG. 17. This test was caried out with the 0-5 kV HV AC, 21 kHz power supply and operated with 1.5 kVᵣₘₛ, 15-25 mAᵣₘₛ, and 10 LPM argon gas flow.

As shown in FIG. 17, showerheads 1 and 2 resulted in plasma initiating in the center of the array. Showerheads 3 and 4 allowed for the full element to turn on, but the CAP required more current for full operation.

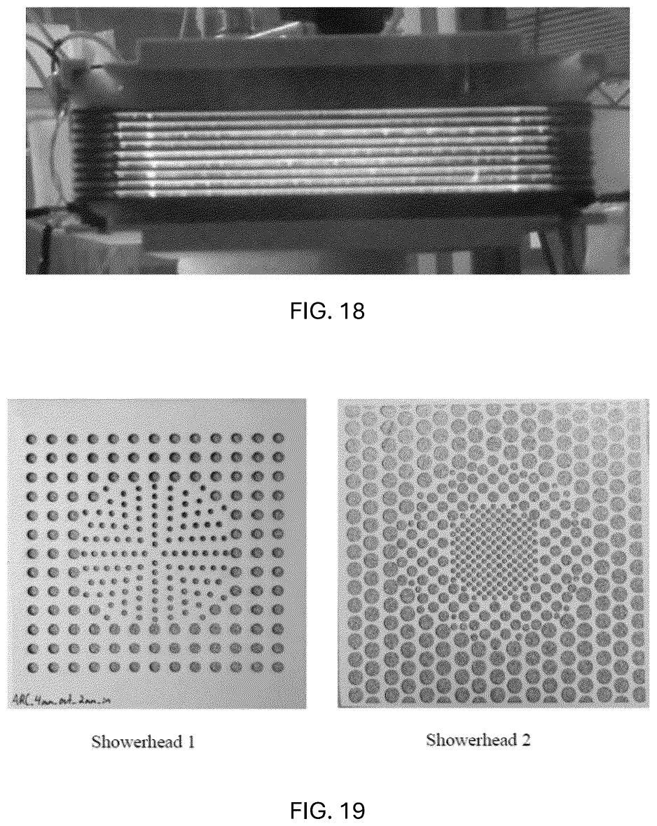

In a subsequent test, a high capacity 0-10 kV, 10-60 kHz HV AC power supply was used, and this power supply was capable of driving a higher load capacitance at higher current (>500 mA). FIG. 18 shows the result of the device assembled with showerhead 4 and tested with 800 Vᵣₘₛ, 40 mAᵣₘₛ, and 30 LPM argon gas using the larger power supply. It was demonstrated that the device required higher current along with higher gas flow rates to turn on all the elements. This power supply was then used for all further experiments. The device shown in FIG. 18 was operated with a 200-pF capacitor connected in parallel. The power supply was designed to operate at higher capacitance load (>100 pF). Because of the low capacitance of the smaller number of elements, the parallel capacitor had to be added to achieve resonance.

Larger devices with showerhead were tested. Two exemplary showerheads are shown in FIG. 19. To observe the effect of showerheads on gas distribution, the gas velocity was measured throughout the device using different showerheads. A CLIMOMASTER 6501-0E anemometer was used to measure gas velocity. The gas velocity was measured at 25 spots across the array, in a 5×5 grid. Table 1 provides the gas velocity (m/s) measured with showerhead 1 in FIG. 19 and Table 2 represents the gas velocity with showerhead 2 in FIG. 19. The gas is well distributed with showerhead 1, and therefore showerhead 1 was selected for further experimentation.

TABLE 1
Gas velocity (m/s) measurement of showerhead 1.

| | | | | |
|---|---|---|---|---|
| 0.33 | 0.32 | 0.44 | .044 | 0.44 |
| 0.31 | 0.31 | 0.33 | 0.41 | 0.40 |
| 0.33 | 0.31 | 0.29 | 0.37 | 0.44 |
| 0.35 | 0.33 | 0.33 | 0.42 | 0.44 |
| 0.35 | 0.35 | 0.42 | 0.42 | 0.47 |

TABLE 2
Gas velocity (m/s) measurement of showerhead 2.

| | | | | |
|---|---|---|---|---|
| 0.24 | 0.14 | 0.07 | 0.14 | 0.14 |
| 0.29 | 0.25 | 0.11 | 0.17 | 0.39 |
| 0.8 | 1.4 | 1.8 | 1.22 | 0.45 |
| 0.22 | 0.14 | 0.37 | 0.27 | 0.43 |
| 0.21 | 0.31 | 0.31 | 0.36 | 0.46 |

### Example 2—Evaluating the Electrical Connection of the Array

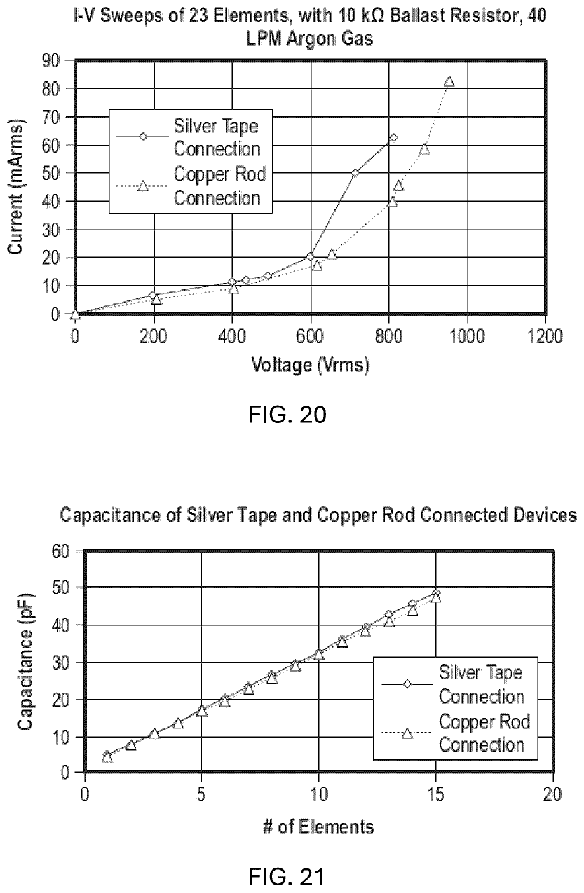

In this Example, two CAP devices of 23 elements of plates according to version 2.1 and 3.1 in FIG. 13 were assembled and tested to observe the difference between the two versions. Version 2.1 utilizes a silver tape connection as shown in FIG. 15 while version 3.1 utilizes 2.3 mm copper rods as depicted in FIG. 16. I-V sweeps of both devices were conducted as shown in FIG. 20. It can be seen that the 3.1 version copper rod connected device works more uniformly than the 2.1 version device due to improved electrical connection between discharge plates. The copper rod connected device operated at higher voltage and current (955 Vᵣₘₛ and 3.59 mAᵣₘₛ per element) than the silver tape connected device (812 Vᵣₘₛ and 2.71 mAᵣₘₛ per element). The operating frequency of the devices were different due to the poor connectivity of the silver tape connection resulting in a difficulty operating at the resonant frequency.

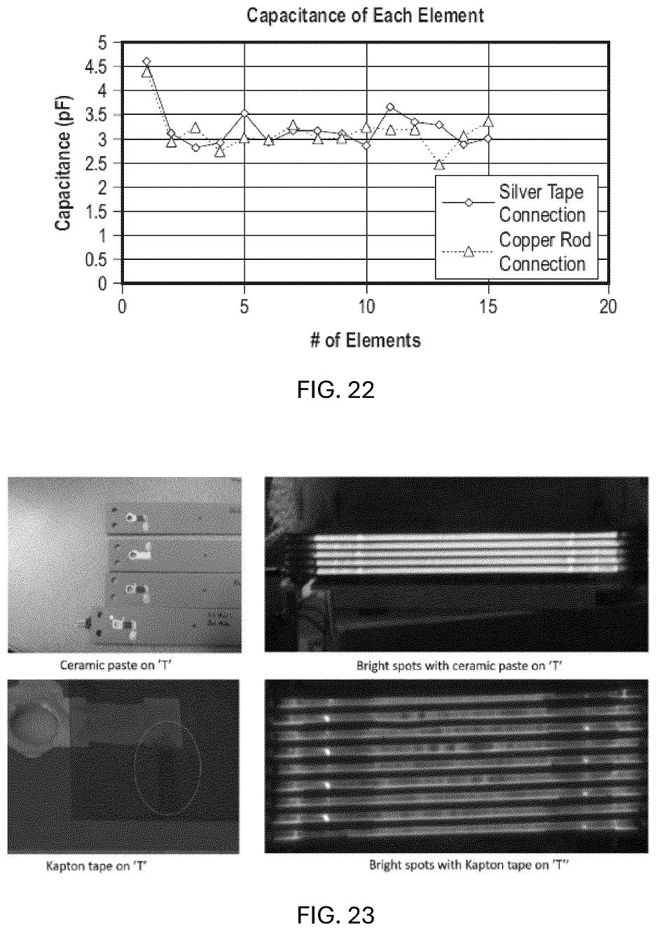

A 43-plate device was then tested. As the device was being assembled, the capacitance measurement of each element was performed. The capacitance of the device is important because it determines the resonant frequency of the array as well as the requirements for the power supply. If each element of the device has significant different capacitance, it will have different operating conditions for each element, leading to a non-uniform device. A comparison of capacitance of each element for both a silver tape connected device and a rod connected device was performed. Results are presented in FIG. 21. Both devices have about the same average capacitance of around 3 pF per element, which ensures that the device can run at resistive limit at resonant frequency. The connection type does not influence the device capacitive properties. The capacitance of individual elements was measured by adding one element at a time as shown in FIG. 22. However, the higher capacitance value of the first element is due to the connection giving the first element a capacitance of 4.5 pF for both cases.

### Example 3—Evaluating Spacer Dimensions

In this Example, an experiment to observe the device behavior for different spacer dimensions was conducted, where spacers of 1.1 mm and 0.55 mm were tested, in a 2-element array. With a 0.55 mm spacer, the plasma is very uniform and saturated, with a 1.1 mm spacer, the plasma is non-uniform and has multiple filaments. Although a 0.55 mm spacer works well, it is not very practical to use since it would require a much larger number of discharge elements to cover an equivalent area. 1.5 mm and 2 mm spacers were also tested. However, the devices could not be turned on with the thicker discharge gaps. Much higher voltage (>3 kVᵣₘₛ) needs to be applied to generate enough electric field for devices with the thicker discharge gaps. Operating at higher voltage can damage the discharge plates as pin holes form on the electrode dielectric from arcs.

### Example 4—Evaluating Device Geometry and Plasma Uniformity

In this Example, variations on the device geometry were studied. It was found that the “T” line circled on versions 1.1-3.1 of the discharge plates shown in FIG. 13 was coupling to the electrode on the adjacent plate and forming a plasma diagonally creating intense bright spots. The “T” line is where the via electrically connects to the discharge electrode.

To prevent bright spots, the “T” junction was covered with ceramic paste to make it harder to form dielectric barrier discharges on that spots. However, even with several layers of ceramic paste, the bright spots persisted. Next, multiple layers of Kapton tape was applied to the “T” junction and was unsuccessful. Even with seven layers of Kapton tape, the bright spots persisted. The results are shown in FIG. 23.

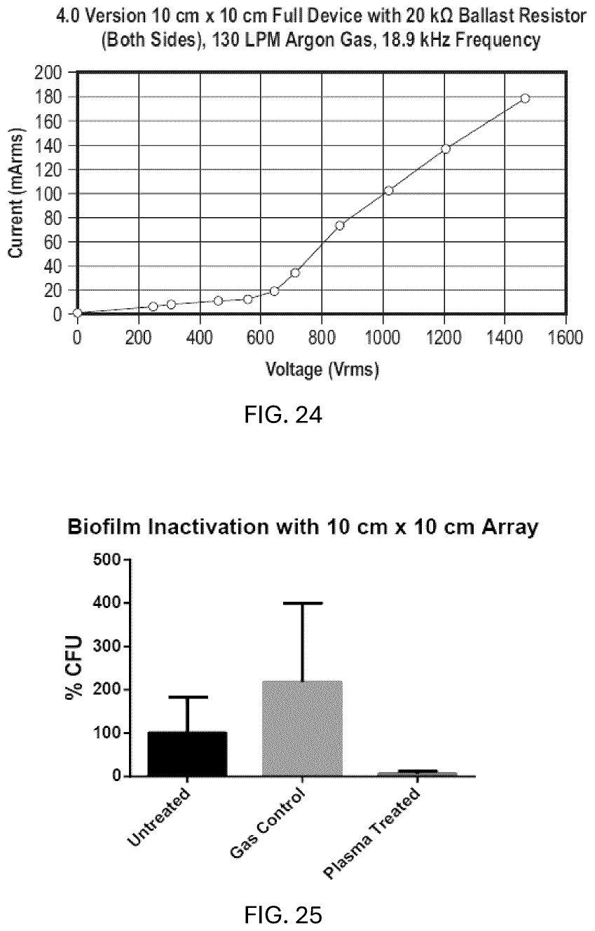

The “T” junction was then covered with the Delrin spacer, which proved successful in preventing bright spots as shown in FIG. 24. No bright spots were observed, however the spacer created a blank region of no plasma in the plasma array.

The discharge plates were then re-designed eliminating the “T” junction. This redesign is shown in FIG. 13 version 3.4 and 4.0. The “T” junction is removed and the electrode is printed to connect to the end, eliminating the “T”.

### Example 5—Characterizing a 10 cm by 10 cm Array

A device of 43 discharge elements was tested with version 3.4 discharge plate (see FIG. 3) with a 10 kΩ ballast resister on both sides. This device was tested with 120 LPM Argon gas flow, 980 Vᵣₘₛ, 120.6 mAᵣₘₛ and 23 kHz frequency. This device showed filamentary discharges that move in time and are typical of CAP discharges.

An I-V sweep was conducted with the device, and the device was not operating at expected operating parameters (>1 kV). The 10 kΩ ballast resistor was not large enough to allow device operation at a high enough voltage to achieve good plasma uniformity. The voltage could not be increased higher than 980 Vᵣₘₛ due to arc formation. Because the ballast resistor did not limit enough current flowing through each element, some elements would develop very high current filaments which traversed across multiple elements creating arcs. These arcs would destroy the device over time if the device was not turned off.

Although the device turned on without any issues, the operating voltage was very low (<1 kVᵣₘₛ). The voltage needed to be increased to run the device at resistive limit. As a 10 kΩ ballast resistor was not sufficient in controlling current to each element, higher ballast resistors (15 kΩ and 20 kΩ) were tested.

Two 8-element devices of the 3.4 version discharge plates were assembled with 15 kΩ and 20 kΩ ballast resistors. The device with 20 kΩ ballast resistor can operate at a higher voltage (1366 Vᵣₘₛ, 6.41 mAᵣₘₛ per element) than with either the 10 kΩ (980 Vᵣₘₛ, 2.71 mAᵣₘₛ per element) or 15 kΩ (963 Vᵣₘₛ, 5.15 mAᵣₘₛ per element) arrays. The 20 kΩ ballast resistor was selected for further tests.

A 43 element (10 cm×10 cm) plasma array was assembled with 20 kΩ ballast resistors on both sides. There were fewer filaments present and the plasma was more uniform. However, lines of filaments formed on both sides of the device near the spacers. Therefore, the placement of the connecting copper rods was investigated.

The connecting copper rod was placed in the active region of the device, and it was suspected that these rods disrupted the air flow and caused the lines of filaments. A new version of the device was designed, version 4.0 as shown in FIG. 13. In version 4.0, the connecting rod and resistor pads are moved to the sides, making the active region free of physical disruption, and a new spacer was designed accordingly. A 43 element 10 cm×10 cm device with version 4.0 was assembled and tested, and the result is shown in FIG. 3. This version works as per expectation, and the plasma is more uniform and operating near resistive limit as there are almost no filaments visually observable.

An I-V sweep of the version 4.0 10 cm×10 cm CAP device was conducted and is presented in FIG. 24. The device is operated with 130 LPM of Argon gas at 18.9 kHz resonant frequency. The device runs at resistive limit creating uniform plasma, and it is evident from the I-V curve where the current-voltage slope is approximately linear.

### Example 6—Testing Effectiveness for Inactivating Biofilm

The biofilm tested with this device was Pseudomonas fluorescens.

The biofilm was prepared as follows. First, the substrates were cut into small coupon shapes (12 mm×12 mm) and then sterilized by cleaning with bleach and then autoclaving. Then the biofilm was grown on the coupons. A culture plate was prepared, where the biofilm culture colonies are grown in LB agar poured into a petri-dish. 2 ml of LB broth is poured into a 30 ml tube. One bacterial colony was taken from the culture plate with a sterile inoculating loop and mixed with the LB broth. The cap of the tube was closed loosely to allow air flow inside. The tube was then kept on a shaker table for 1 day at 170 RPM. The sample coupons were sterilized at this stage. After 1 day, the tube with bacterial seed culture was taken out of shaker table and a dilution was made with the LB broth and the culture at 50:1 ratio and vortexed to mix. The coupons were placed in a 12 well plate and a 2 ml dilution of the bacterial culture was poured in each well containing the coupons. Then the 12 well plate was sealed with parafilm and placed on the shaker table for 4 days at 170 RPM.

For testing, the coupons were taken out with a tweezer and dipped in DI water. The edges were cleaned with a cotton swab dipped in IPA. The back of the coupon was cleaned with a paper towel soaked in IPA. A petri-dish with filter paper soaked in PBS was prepared, and the cleaned coupons were placed in the petri-dish.

The device was then set up and the coupons placed on the stage under the device. The device was operated for a predetermined time exposing the coupons to the plasma. The coupons were removed and placed in the petri-dish again. The petri-dish was labeled with treatment type and treatment time. The coupons sat for 90 minutes after treatment. After the coupons were treated and allowed to rest, they were placed in 50 ml conical tubes with glass beads and 10 ml PBS. Then the tubes were vortexed for 3 minutes to disaggregate the biofilm into the solution and liberate individual bacterial cells. This solution is called neat. The neat is then diluted in different ratios with PBS and plated on LB agar plates.

For each coupon, 3 dilutions were prepared with neat solution and PBS at 1:100, 1:1000, and 1:10000 ratios. For each dilution 2 volumes (10 μl and 100 μl) of solution were delivered to the surface of agar plates containing 4 mm diameter sterile glass beads. After adding solution to the agar plates, the plate was shaken for 20 seconds for the glass beads to distribute the bacterial inoculum, and then the glass beads were removed. The plates were stacked and kept in an incubator for at least 24 hours. After at least 24 hours, the colonies were grown in a petri-dish, and the colony forming units (CFUs) were counted and analyzed to determine the inactivation percentage. In these studies, a CFU is equal to a single bacterial cell found in the initial inoculum placed on the agar plate.

A 43-plate 10 cm×10 cm array was tested using the following parameters: 1.15 kVᵣₘₛ voltage, 138 mAᵣₘₛ current, 20 KHz frequency, 130 LPM argon and 5 LPM oxygen, 20 kΩ ballast resistors, a distance from device to substrate of 15 mm, and a treatment time of 2.5 minutes.

Three samples were tested for each treatment type. After 24 hours incubation time, colony forming units (CFUs) were counted and analyzed. The inactivation result was 84% CFU reduction in the plasma treated bacterial biofilm.

Another test was conducted with an increased treatment time. This time the inactivation rate increased to a 94% reduction of the CFU in the plasma treated bacterial biofilm as shown in FIG. 25. The parameters were as follows: 1.25 kVᵣₘₛ voltage, 130 mAᵣₘₛ current, 19 kHz frequency, Ar (130 LPM) and 02 (5 LPM), 3 minutes treatment time, 30 mm substrate distance, 90 minute resting time.

Next surface sanitation tests were conducted by growing biofilm on high-pressure laminate coupons. The parameters were as follows: 1.5 kVᵣₘₛ voltage, 160 mAᵣₘₛ current, 19 kHz frequency, Ar (130 LPM) and 02 (5 LPM), 150 second treatment time, 15 mm substrate distance, 90 minute resting time.

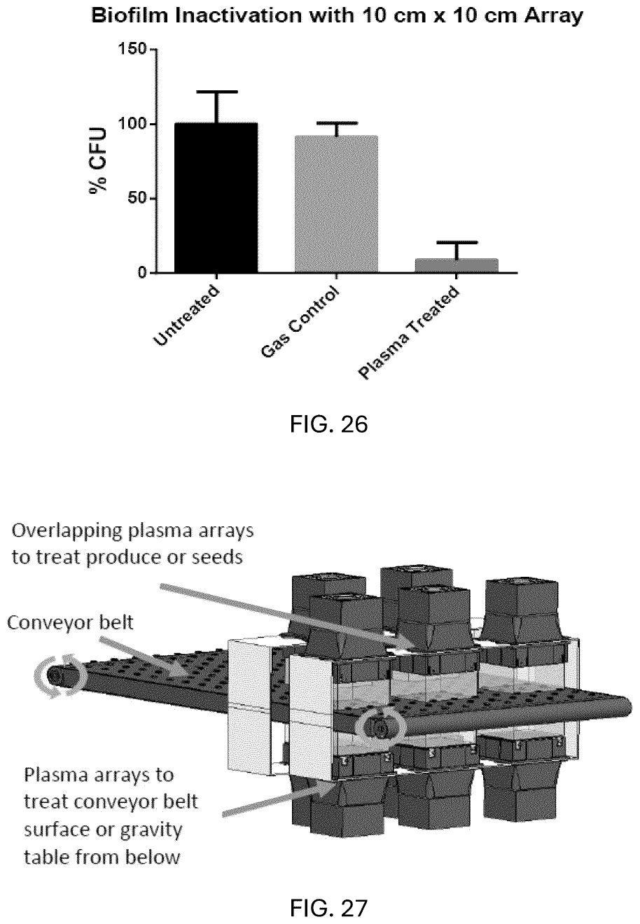

Good inactivation was obtained with the high-pressure laminate as shown in FIG. 26. In this case, the gas control inactivated only 8% of bacterial cells in the biofilm, and the plasma treatment inactivated more than 91% of the bacterial biofilm cells.

### Example 7—Multi-Cap Array

In this example, several large CAP arrays based on the 10 cm×10 cm device are grouped together. Shown in FIG. 11 is an illustration showing 5 CAP arrays combined to treat a conveyor belt or other separating/selection surface from the bottom of the surface as it rotates or vibrates. This orientation could provide a clean-in-place (CIP) solution during operation, providing a constant sanitation that could prevent biofilm accumulation or pathogen transfer on a conveyor belt surface. Alternatively, if the surface is porous such as used in air-assisted vibrating gravity tables used in seed selection, placement of the arrays below the table would position the plasma in an airstream to accomplish both seed sanitization and density separation. Also shown are 5 staggered CAP arrays oriented above the conveyor belt that could be used to simultaneously treat produce (fresh fruit, seeds, etc.) prior to packaging as well as the conveyor surface.

In this example, 5 CAP arrays are positioned at the beginning (return) of the conveyor belt as shown in FIG. 11. These arrays treat an approximately 35-40 cm wide conveyor belt in a staggered formation. As each array has a non-active region (bezel), the arrays have some overlap to ensure full surface treatment. As the belt surface passes through the continuously operating arrays, the plasma reactive species inactivates and removes pathogens from the surface. The technology can be utilized for a variety of conveyor belt sizes by simply increasing the number of CAP arrays employed.

The same conveyor belt system can be used to treat crop seeds (such as sweetcorn) and/or produce. As shown in FIG. 11, the seeds or produce can be placed on the conveyor and moved under a staggered array of 5 CAP devices (the number of CAP devices in an array can vary based on the size of the conveyor). Alternatively, seeds are often processed on a density table separation to remove higher density rocks and lower density chaff and defective seeds by agitating over airflow that travels from below through the angled table surface. In the process, seeds turn over so the whole surface can be exposed to plasma treatment.

### Example 8—Air as the Working Gas, Exhaust and Recycle Systems

Systems using 10 cm×10 cm CAP arrays with air as the working gas will be developed. Arrays with air as the working gas are more difficult to operate requiring high AC voltage to achieve uniform plasma. The arrays may require 2.5 kV, and power dissipation will be high (300-400 W/array). Therefore, a new plasma driver system and array cooling may be required in addition to a system providing operation of multiple arrays continuously. This system may operate continuously for days while not overheating.

The systems produce reactive oxygen and nitrogen species (RONS) needed to inactivate the pathogens. These species include ozone (O₃) hydrogen peroxide (H₂O₂), singlet oxygen (¹O₂), superoxide (O₂⁻), hydroxyl radical (OH·), nitric oxide (NO), and peroxynitrite (ONOO⁻). These gases must be captured for safety reasons and would normally be exhausted, but they also offer a method for improving the CAP treatment efficacy and for reducing treatment times. This embodiment employs a capture system where each CAP array has a gas return path that provides negative pressure (suction) to pull the RONS back to the array fan inlet to recirculate the RONS back through the air inlet to enhance the CAP treatment of the surface. Recirculating the RONS may reduce treatment times.

A drawing showing the capture system with return pipes is shown in FIG. 7. In this embodiment, the capture structure comprises piping to direct the capture gas to the rear where it is either filtered to remove exhaust gas or recirculated (not shown) into the air injection fan.

### Example 9—Effectiveness on Various Microbial Contaminants and Biofilms

The impact of plasma treatment on microbial contamination and biofilm accumulation on surfaces will be measured using a series of static and in-motion tests. This example will test the effectiveness of a set of viral (Feline Calici Virus, FCV), bacterial (Ps. fluorescens, Ps. syringae, E. coli, S. enterica), and fungal (Fusarium graminareum, Pythium ultimum) species. The virus FCV was chosen since it is non-pathogenic to humans, but is a model of the highly contagious enteric Norovirus that is transmitted through contact with contaminated surfaces. The other bacterial and fungal species selected for this proof of concept studies are either human pathogens that cause foodborne illness (E. coli, S. enterica), spoilage organisms encountered in food processing (Ps. fluorescens), or environmental bacterial or fungal plant pathogens (Ps. syringae, F. graminarium, P. ultimum). The effect of both CAP exposure time and exposure distance on microbial survival will be studied.

### Example 10—Assessing CAP Array Production of RONS

In this Example, the assays for the detection and quantification of six RONS: ozone, hydrogen peroxide, hydroxyl radicals, superoxide, nitric oxide and peroxynitrite, will be established using air as the feedstock gas.

### Example 11—Assessing CAP Array Treatment on Seed Germination

It is not expected that CAP treatment of seeds for the length of time required to inactivate surface pathogens will cause a loss of germination rate (due to the outer coating of the seed), however germination studies will be conducted. These studies will be performed according to standard USDA germination protocols and reflect the plasma exposure conditions required to effect significant (99-99.9%) antimicrobial activity.

The present disclosure is further defined by the following numbered embodiments:

Embodiment 1. A cold atmospheric pressure plasma (CAP) array system comprising: a) a CAP array comprising a discharge element comprising two discharge plates each comprising a conducting electrode covered with a dielectric material, and a spacer configured to define the distance between discharge plates; b) a gas flow inlet configured to flow a working gas through the CAP array; c) a showerhead configured between the gas flow inlet and the CAP array; and d) a conducting rod at each end of the CAP array configured to establish electrical connection to the conducting electrode of each discharge plate; wherein the discharge plates are stacked together and electrically connected in parallel through the conducting rods, and wherein one conducting rod is in electrical connection to ground and the other is in electrical connection with a power supply.

Embodiment 2. The cold atmospheric pressure plasma array system of embodiment 1, wherein the CAP array comprises more than two, more than eight, more than 20, more than 40, or about 43 discharge elements, wherein each discharge plate, excluding two end plates, comprise an electrode on each side of said plate, and wherein the discharge elements are stacked together and electrically connected in parallel through the conducting rods.

Embodiment 3. The cold atmospheric pressure plasma array system of any one of embodiments 1-2, wherein the spacer is a Delrin spacer.

Embodiment 4. The cold atmospheric pressure plasma array system of any one of embodiments 1-3, wherein the spacer is configured such that the space between the discharge plates is about 0.5 mm to about 2 mm, or between about 0.7 mm to about 1.1 mm.

Embodiment 5. The cold atmospheric pressure plasma array system of any one of embodiments 1-4, wherein the discharge plates comprises ceramic and the dielectric material comprises ceramic.

Embodiment 6. The cold atmospheric pressure plasma array system of any one of embodiments 1-5, wherein the discharge plate comprises low-temperature co-fired ceramic and the dielectric material comprises low-temperature co-fired ceramic.

Embodiment 7. The cold atmospheric pressure plasma array system of any one of embodiments 1-6, wherein the conducting rods are electrically connected to the conducting electrode by silver paint.

Embodiment 8. The cold atmospheric pressure plasma array system of any one of embodiments 1-7, wherein the conducting electrode is covered with about 50 μm to about 200 μm of dielectric material.

Embodiment 9. The cold atmospheric pressure plasma array system of any one of embodiments 1-8, wherein the conducting electrode is substantially a straight line across the bottom portion of the discharge plate.

Embodiment 10. The cold atmospheric pressure plasma array system of any one of embodiments 1-9, wherein the conducting electrode comprises silver.

Embodiment 11. The cold atmospheric pressure plasma array system of any one of embodiments 1-10, wherein discharge plates further comprise a ballast resistor in electrical connection with the conducting electrode.

Embodiment 12. The cold atmospheric pressure plasma array system of embodiment 11, wherein the ballast resistor is connected to the conducting electrode with silver paint.

Embodiment 13. The cold atmospheric pressure plasma array system of any one of embodiments 11-12, wherein the ballast resistor comprises a resistance of from about 10 kΩ to about 20 kΩ. In an embodiment, the ballast resistance is about 20 kΩ, or at least about 20 kΩ.

Embodiment 14. The cold atmospheric pressure plasma array system of any one of embodiments 11-13, wherein the ballast resistors of two adjacent plates are on opposite ends of the plates, connected to electrodes at different potentials.

Embodiment 15. The cold atmospheric pressure plasma array system of any one of embodiments 1-14, wherein the discharge plate is configured such that the conducting rod is not in the active region of the discharge plate.

Embodiment 16. The cold atmospheric pressure plasma array system of any one of embodiments 1-15, wherein the conducting electrode does not comprise a right angle and is connected to the ballast resistor and/or conducting rod with a curved shape.

Embodiment 17. The cold atmospheric pressure plasma array system of any one of embodiments 1-16, wherein the system comprises a nonconducting threaded assembly rod at each end of the array configured to hold the assembled CAP array together.

Embodiment 18. The cold atmospheric pressure plasma array system of any one of embodiments 1-18, wherein the showerhead comprises round holes that are not uniform in size or distribution, wherein the showerhead comprises smaller holes in the center half of the showerhead.

Embodiment 19. The cold atmospheric pressure plasma array system of embodiment 18, wherein the showerhead comprises holes with about 4 mm diameter in the outer ⅓ to outer ⅔ of the showerhead surface and holes with a diameter of about 2 mm in the inner ⅓ to inner ⅔ of the surface area of the showerhead.

Embodiment 20. The cold atmospheric pressure plasma array system of any one of embodiments 1-19, further comprising an enclosure configured to house the CAP array, the showerhead, the gas inlet.

Embodiment 21. The cold atmospheric pressure plasma array system of any one of embodiments 1-20, wherein the gas flow inlet comprises a fan and the working gas comprises air.

Embodiment 22. The cold atmospheric pressure plasma array system of any one of embodiments 1-21, wherein the system further comprises a capture system configured to capture gas effluent.

Embodiment 23. The cold atmospheric pressure plasma array system of embodiment 22, wherein the capture system comprises a negative pressure region.

Embodiment 24. The cold atmospheric pressure plasma array system of any one of embodiments 22-23, wherein the capture system comprises a fan to provide suction.

Embodiment 25. The cold atmospheric pressure plasma array system of any one of embodiments 22-24, wherein the gas flow inlet comprises a fan, and said fan is further configured to provide suction for the capture system.

Embodiment 26. The cold atmospheric pressure plasma array system of any one of embodiments 22-25, wherein the capture system comprises an exhaust system and/or filtration configured to remove pathogenic detritus from the system.

Embodiment 27. The cold atmospheric pressure plasma array system of any one of embodiments 22-26, wherein the capture system comprises a material that contacts at least a portion of the treatment surface.

Embodiment 28. The cold atmospheric pressure plasma array system of any one of embodiments 22-27, wherein the capture system comprises a material that contacts at least a portion of the treatment surface configured to form a closed system to prevent gas species release.

Embodiment 29. The cold atmospheric pressure plasma array system of any one of embodiments 27-28, wherein the material is fibrous.

Embodiment 30. The cold atmospheric pressure plasma array system of any one of embodiments 22-29, wherein the capture system comprises a gas return path configured to recirculate the effluent back to the gas inlet.

Embodiment 31. The cold atmospheric pressure plasma array system of any one of embodiments 22-30, wherein the gas capture system is adjacent to at least one side of the CAP array.

Embodiment 32. The cold atmospheric pressure plasma array system of any one of embodiments 22-31, wherein the gas capture system surrounds the CAP array on all sides.

Embodiment 33. The cold atmospheric pressure plasma array system of any one of embodiments 1-32, comprising a current transformer configured for electrical connection with the power supply and a conducting rod.

Embodiment 34. The cold atmospheric pressure plasma array system of any one of embodiments 1-33, wherein the system further comprises a cooling device and/or cooling system.

Embodiment 35. The cold atmospheric pressure plasma array system of embodiment 34, wherein the cooling system comprises a polymer at the edges of the CAP array elements configured to pull heat away from the array and ballast resistors.

Embodiment 36. The cold atmospheric pressure plasma array system of embodiment 34, wherein the cooling system comprises spacers with high thermal conductivity.

Embodiment 37. The cold atmospheric pressure plasma array system of embodiment 34, wherein the cooling system comprises copper plates configured to trap polymer against the array, and these plates extend back behind the array to convective cooling fins or a conductive water coolant system.

Embodiment 38. The cold atmospheric pressure plasma array system of any one of embodiments 1-37, further comprising a plasma driver.

Embodiment 39. The cold atmospheric pressure plasma array system of embodiment 38, wherein the working gas comprises air and the driver is configured to allow operation at high voltages and current.

Embodiment 40. The cold atmospheric pressure plasma array system of any one of embodiments 38-39, wherein the plasma driver is configured to allow programmed control to autotune the resonant frequency after plasma initiation.

Embodiment 41. The cold atmospheric pressure plasma array system of any one of embodiments 38-40, wherein the driver comprises a controller, start and/or shut-down software, current and/or temperature sensors, shut down circuitry, and combinations thereof.

Embodiment 42. A method of treating a surface comprising: applying voltage to and flowing a working gas through, the cold atmospheric pressure plasma array system of any one of embodiments 1-41 and exposing a surface to a plasma generated by the cold atmospheric pressure plasma array system.

Embodiment 43. The method of embodiment 42, wherein the plasma comprises reactive oxygen and nitrogen species.

Embodiment 44. The method of any one of embodiments 42-43, wherein the working gas comprises argon, nitrogen, oxygen, ambient air, or combinations thereof.

Embodiment 45. The method of any one of embodiments 42-44, wherein the flow rate of the working gas is from about 1 LPM to about 300 LPM.

Embodiment 46. The method of any one of embodiments 42-45, wherein the voltage is from about 0.9 kVᵣₘₛ to about 5.0 kV.

Embodiment 47. The method of any one of embodiments 42-46, wherein the working gas is air and the and the voltage is from about 2 kV to about 3 kV, or from about 1 kV to about 2.5 kV.

Embodiment 48. The method of any one of embodiments 42-47, wherein the surface is exposed to the plasma for about 150 seconds to about 5 minutes, or longer.

Embodiment 49. The method of any one of embodiments 42-48, wherein the surface is from about 5 mm to about 100 mm from the CAP array, or from about 15 mm to about 30 mm from the CAP array.

Embodiment 50. The method of any one of embodiments 42-49, wherein the method comprises more than one cold atmospheric pressure plasma array system.

Embodiment 51. The method of any one of embodiments 42-50, wherein the method results in colony forming unit (CFU) reduction of at least about 50%, at least about 60%, at least about 70%, at least about 80%, at least about 90%, at least about 90%, or about 100%.

Embodiment 52. A gas capture system comprising: a) a CAP plasma array configured to form a plasma, and b) a capture system configured to capture gas effluent during and after plasma formation.

Embodiment 53. The gas capture system of embodiment 52, wherein the capture system comprises a negative pressure region.

Embodiment 54. The gas capture system of any one of embodiments 52-53, wherein the capture system comprises a fan to provide suction.

Embodiment 55. The gas capture system of any one of embodiments 52-54, wherein the gas flow inlet comprises a fan, and said fan is further configured to provide suction for the capture system.

Embodiment 56. The gas capture system of any one of embodiments 52-55, wherein the capture system comprises an exhaust system and/or filtration configured to remove pathogenic detritus from the system.

Embodiment 57. The gas capture system of any one of embodiments 52-56, wherein the comprises a material that contacts at least a portion of the treatment surface.

Embodiment 58. The gas capture system of any one of embodiments 52-57, wherein the comprises a material that contacts at least a portion of the treatment surface configured to form a closed system to prevent gas species release.

Embodiment 59. The gas capture system of any one of embodiments 57-58, wherein the material is fibrous.

Embodiment 60. The gas capture system of any one of embodiments 52-60, wherein the capture system comprises a gas return path configured to recirculate the effluent back to a gas inlet.

Embodiment 61. The gas capture system of any one of embodiments 52-60, wherein the gas capture system is adjacent to at least one side of the CAP plasma array.

Embodiment 62. The gas capture system of any one of embodiments 52-61, wherein the gas capture system surrounds the CAP plasma array on all sides.

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

What is claimed is:

1. A cold atmospheric pressure plasma (CAP) array system comprising:
a) a CAP array comprising a discharge element comprising two discharge plates each comprising a conducting electrode covered with a dielectric material, and a dielectric spacer configured to define the distance between discharge plates;
b) a gas flow inlet configured to flow a working gas through the CAP array;
c) a showerhead configured between the gas flow inlet and the CAP array; and
d) a conducting rod at each end of the CAP array configured to establish electrical connection to the conducting electrode of each discharge plate;
wherein the discharge plates are stacked together and electrically connected in parallel through the conducting rods, and
wherein one conducting rod is in electrical connection with a power supply and the other is in electrical connection with a power supply or to ground.

2. The cold atmospheric pressure plasma array system of claim 1, wherein the CAP array comprises more than two, more than eight, more than 20, more than 40, or about 43 discharge elements, wherein each discharge plate, excluding two end plates, comprise an electrode on each side of said plate, and wherein the discharge elements are stacked together and electrically connected in parallel through the conducting rods.

3. The cold atmospheric pressure plasma array system of claim 1, wherein the spacer is configured such that the space between the discharge plates is about 0.5 mm to about 2 mm, or between about 0.7 mm to about 1.1 mm.

4. The cold atmospheric pressure plasma array system of claim 1, wherein the discharge plates comprises ceramic and the dielectric material comprises ceramic.

5. The cold atmospheric pressure plasma array system of claim 1, wherein the conducting rods are electrically connected to the conducting electrode by silver paint and/or solder, and wherein the conducting electrode is covered with about 50 μm to about 200 μm of dielectric material.

6. The cold atmospheric pressure plasma array system of claim 1, wherein the conducting electrode is substantially a straight line across the bottom portion of the discharge plate, and wherein the conducting electrode comprises silver.

7. The cold atmospheric pressure plasma array system of claim 1, wherein discharge plates further comprise a ballast resistor or a capacitor in electrical connection with the conducting electrode, wherein the ballast resistor is connected to the conducting electrode with silver paint and/or solder, and wherein the ballast resistors of two adjacent plates are on opposite ends of the plates, connected to electrodes at different potentials.

8. The cold atmospheric pressure plasma array system of claim 1, wherein the conducting electrode does not comprise a right angle and is connected to the ballast resistor and/or conducting rod with a curved shape.

9. The cold atmospheric pressure plasma array system of claim 1, wherein the system comprises a nonconducting threaded assembly rod at each end of the array configured to hold the assembled CAP array together.

10. The cold atmospheric pressure plasma array system of claim 1, wherein the showerhead comprises round holes that are not uniform in size or distribution, wherein the showerhead comprises smaller holes in the center half of the showerhead.

11. The cold atmospheric pressure plasma array system of claim 1, further comprising an enclosure configured to house the CAP array, the showerhead, the gas inlet, wherein the gas flow inlet comprises a fan and the working gas comprises air.

12. The cold atmospheric pressure plasma array system of claim 1, wherein the system further comprises a capture system adjacent to at least one side of the CAP array configured to capture gas effluent, wherein the capture system comprises a negative pressure region, and wherein the capture system comprises an exhaust system and/or filtration configured to remove pathogenic detritus from the system.

13. The cold atmospheric pressure plasma array system of claim 12, wherein the capture system comprises a gas return path configured to recirculate the effluent back to the gas inlet.

14. The cold atmospheric pressure plasma array system of claim 1, wherein the system further comprises a cooling device and/or cooling system.

15. The cold atmospheric pressure plasma array system of claim 14, wherein the cooling system comprises a polymer at the edges of the CAP array elements configured to pull heat away from the array and ballast resistors, or wherein the cooling system comprises spacers with high thermal conductivity, or wherein the cooling system comprises copper plates configured to trap polymer against the array, and these plates extend back behind the array to convective cooling fins or a conductive water coolant system.

16. The cold atmospheric pressure plasma array system of claim 1, further comprising a plasma driver, wherein the working gas comprises air and the driver is configured to allow operation at high voltages and current, and/or wherein the plasma driver is configured to allow programmed control to autotune the resonant frequency after plasma initiation, and/or wherein the driver comprises a controller, start and/or shut-down software, current and/or temperature sensors, shut down circuitry, and combinations thereof.

17. A method of treating a surface comprising:
applying voltage to and flowing a working gas through the cold atmospheric pressure plasma array system of claim 1; and
exposing a surface to a plasma generated by the cold atmospheric pressure plasma array system,
wherein the plasma comprises reactive oxygen and nitrogen species and wherein the working gas comprises argon, nitrogen, oxygen, ambient air, or combinations thereof, and
wherein the method results in colony forming unit (CFU) reduction of at least about 50%, at least about 60%, at least about 70%, at least about 80%, at least about 90%, at least about 90%, or about 100%.

18. The method of claim 17, wherein the surface is from about 5 mm to about 100 mm from the CAP array, wherein the surface is exposed to the plasma for about 150 seconds to about 5 minutes, wherein the flow rate of the working gas is from about 1 LPM to about 300 LPM, and/or wherein the voltage is from about 0.9 kVᵣₘₛ to about 5.0 kV.

19. The method of claim 17, wherein the working gas is air and the and the voltage is from about 2 kV to about 3 kV.

20. The method of claim 17, wherein the method comprises more than one cold atmospheric pressure plasma array system.
